(19) United States
(12) Patent Application Publication
(10) Pub. No.: US 2011/0009685 A1
(43) Pub. Date: Jan. 13, 2011

(54) MFI Molecular Sieve Composition and the Method of Making the Same

(76) Inventors: Sebastien Kremer, Woluwe-Saint-Lambert (BE); Brita Engels, Begijnendijk (BE); Els Cecilia De Clerck, Aarschot (BE); Katleen Hermans, Herent (BE)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 12/919,706

(22) PCT Filed: Feb. 13, 2009
(86) PCT No.: PCT/US09/34072

§ 371 Date: Aug. 26, 2010

Related U.S. Application Data
(60) Provisional application No. 61/040,955, filed on Mar. 31, 2008.

(51) Int. Cl. C07C 7/20; C01B 39/04; C01B 39/02

(52) U.S. Cl. 585/899; 423/702; 423/700; 423/704

(57) ABSTRACT

The disclosure relates to a substantially uniform molecular sieve having MFI topology and quasi parallelepiped morphology. This disclosure also relates to a method of making the crystalline molecular sieve of this disclosure.

### FIELD OF THE INVENTION

The present disclosure relates to a substantially uniform crystalline molecular sieve having MFI topology and substantially uniform quasi parallelepiped morphology. In particular, the MFI molecular sieve of this disclosure is substantially free of crystal twinning.

### BACKGROUND OF THE INVENTION

Molecular sieve materials, both natural and synthetic, have been demonstrated in the past to have catalytic properties for various types of hydrocarbon conversion. Molecular sieves that find application in catalysis include any of the naturally occurring or synthetic crystalline molecular sieves. Examples of these zeolites include large pore zeolites, intermediate pore size zeolites, and small pore zeolites. These zeolites and their isotypes are described in “Atlas of Zeolite Framework Types”, eds. W. H. Meier, D. H. Olson and Ch. Baerlocher, Elsevier, Fifth Edition, 2001. A large pore zeolite generally has a pore size of at least about 7 Å and includes LTL, VFI, MAZ, FAU, OFF, *BEA, and MOR framework type zeolites (IUPAC Commission of Zeolite Nomenclature). Examples of large pore zeolites include mazzite, offretite, zeolite L, VPI-5, zeolite Y, zeolite X, omega, and Beta. An intermediate pore size zeolite generally has a pore size from about 5 Å to less than about 7 Å and includes, for example, MFI, MEL, EUO, MTT, MFS, AEL, AFO, HEU, FER, MWW, and TON framework type zeolites (IUPAC Commission of Zeolite Nomenclature). Examples of intermediate pore size zeolites include ZSM-5, ZSM-11, ZSM-22, MCM-22, silicalite-1, and silicalite-2. A small pore size zeolite has a pore size from about 3 Å to less than about 5 Å and includes, for example, CHA, ERI, KFI, LEV, SOD, and LTA framework type zeolites (IUPAC Commission of Zeolite Nomenclature). Examples of small pore zeolites include ZK-4, ZSM-2, SAPO-34, SAPO-35, ZK-14, SAPO-42, ZK-21, ZK-22, ZK-5, ZK-20, zeolite A, chabazite, zeolite T, gmelinite, ALPO-17, and clinoptilolite.

ZSM-5 is an MFI-type zeolite, usually an aluminosilicate zeolite, which has been found useful as a catalyst in a variety of processes for preparing, converting, separating or purifying organic compounds. The earliest ZSM-5 zeolites were prepared using an organic template in the synthesis mixture which directed the formation of the ZSM-5 structure. Known ZSM-5 zeolites prepared using an organic template generally have a SiO₂/Al₂O₃ molar ratio of at least 60 and frequently considerably greater than 60 (see e.g. U.S. Pat. No. 4,797,267). So-called “inorganic” ZSM-5 zeolites, made in the absence of an organic template, were prepared in the 1980's. Typically these have a SiO₂/Al₂O₃ molar ratio of from 20 to about 30 to 40. SiO₂/Al₂O₃ ratios of up to 80 have been reported (e.g., Zeolites 1989 Vol. 9, 363-370).

The morphology of ZSM-5 crystals produced using an organic template can vary. For example, large elongated hexagonal prisms, whose corners may be rounded, was reported (see Studies in Surface Science and Catalysis 33, “Synthesis of High Silica Aluminosilicate Zeolites” (Elsevier), P. A. Jacobs and J. A. Martens). Crystals of ZSM-5 which are agglomerates of smaller, elementary hexagonal crystallites are also illustrated. In the presence of an extremely high proportion of silica (e.g., U.S. Pat. No. 4,797,267), ZSM-5 crystals may be rod shaped, i.e., elongated crystals with substantially parallel sides and blunt ends. The morphology of inorganic ZSM-5 crystals tends to be ellipsoidal (Zeolites 1989 Vol. 9, 363-370).

Researchers at Sogang University, Korea (e.g., J. Am. Chem. Soc., 2001, 123, 9769) reported synthesis of relative uniform ZSM-5. ZSM-5 with average crystal size of 2.0×1.5×0.8 μm³ (large ZSM-5) was synthesized from a gel with molar ratios of TEOS:TPAOH:NaAlO₂:H₂O=10:1:0.05:600, where TEOS and TPAOH represent tetraethylorthosilicate and tetrapropylammonium hydroxide, respectively, and the composition of NaAlO₂ was Na₂O=31-35% and Al₂O₃=34-39%. Also, ZSM-5 with average crystal size of 1.0×0.7×0.4 μm³ (medium ZSM-5) was synthesized from a gel with molar ratios of TEOS:TPAOH:NaAl₂:H₂O=6:1:0.05:460 and ZSM-5 with average crystal size of 0.3×0.2×0.1 μm³ (small ZSM-5) was synthesized from a gel with molar ratios of TEOS:TPAOH:NaAlO₂:H₂O=7:1:0.06:280. The ZSM-5 crystals as reported by researchers at Sogang University have roughly the same shape and similar size.

However, the ZSM-5 zeolite disclosed by researchers at Sogang University has limited crystal size and low uniformity. It is well known that uniform crystal size and/or morphology of molecular sieves is often desirable to obtain superior performance in hydrocarbon conversion or separation processes, in film and coating applications and in macrostructure assembly. MFI crystals prepared following standard synthesis methods as disclosed in prior art have a broad size distribution and less discrete morphology, which may potentially limit their performance in the applications requiring uniformity of crystals. There is a need to develop molecular sieve or zeolite with high uniformity in both size and morphology.

The applicants have now identified a novel form of molecular sieve having MFI topology, such as, ZSM-5 and/or silicalite-1. The molecular sieve of the present invention are substantially uniform and have quasi parallelepiped morphology. In particular, the crystals of the MFI molecular sieve of this disclosure are substantially free of crystal twinning.

### DESCRIPTION OF FIGURES

FIG. 1a is a SEM picture of the product of Example 29.

FIG. 1b illustrates a top view of a molecular sieve crystal of this invention.

FIG. 1c illustrates a side view of a molecular sieve crystal of this invention.

FIG. 2 is a SEM picture of the product of Example 6.

FIG. 3 is a SEM picture of the product of Example 36.

FIG. 4 is a SEM picture of the product of Example 18.

FIG. 5 is a SEM picture of the product of Example 18.

### SUMMARY OF THE INVENTION

The disclosure relates to a crystalline molecular sieve having MFI topology comprising substantially uniform sized crystal having substantially uniform quasi parallelepiped morphology.

In some aspects, the molecular sieve is substantially free of crystal twinning. In other aspects, the molecular sieve is substantially flat measured by SEM. In yet other aspects, the length of the molecular sieve crystal is at least 1 μm (micrometer).

In some embodiments, the molecular sieve of this disclosure is an aluminosilicate ZSM-5 molecular sieve or silicalite-1.

In some aspects, the molecular sieve of this disclosure has a uniformity of crystal size of 90%±10% when measured by SEM or a span of less than 3 measured by laser scattering. In other aspects, the molecular sieve of this disclosure has a uniformity of L/H and/or a uniformity of L/W of 90%±10% measured by SEM.

In some embodiments, this disclosure relates to a method of making a crystalline molecular sieve of this disclosure, the method comprises:
(a) providing a mixture comprising at least one source of at least one tetravalent element (Y), preferably silicon; at least one source of hydroxide ion, at least one directing-agent (R), water, optionally at least one source of at least one trivalent element (X), preferably aluminum; optionally at least one source of at least one metal element (M), preferably alkali metal or alkali earth metal; the mixture having the following molar composition:
   Y/X₂=10 to infinity, preferably 50 to 10000, more preferably 500 to 600
   H₂O/Y=10 to 1000, preferably 20 to 200
   M/Y=0 to 0.5, preferably 0 to 0.2
   OH⁻/Y=0.001 to 2, preferably 0.05 to 2, more preferably 0.1 to 1
   R/Y=0.001 to 2, preferably 0.05 to 2
   wherein R is TPA, wherein OH⁻/Y is not corrected for trivalent ion;
(b) submitting the mixture at crystallization conditions to form a product comprising the desired crystalline molecular sieve, wherein the crystallization conditions comprise a temperature in the range of from 120° C. to 250° C., a crystallization time from about 1 hour to 200 hours; a heating rate in the range from at least 20° C./h, and a stirring speed at least 50 RPM, prefefably at least 100 RPM and less than 600 RPM; and
(c) recovering the molecular sieve.

In other embodiments, this disclosure relates to a process for hydrocarbon conversion, comprising the step of contacting a hydrocarbon feedstock with the crystalline molecular sieve of this disclosure, under conversion conditions to form a conversion product.

### DETAILED DESCRIPTION OF THE INVENTION

### Introduction

All patents, patent applications, test procedures, priority documents, articles, publications, manuals, and other documents cited herein are fully incorporated by reference to the extent such disclosure is not inconsistent with the present invention and for all jurisdictions in which such incorporation is permitted.

When numerical lower limits and numerical upper limits are listed herein, ranges from any lower limit to any upper limit are contemplated.

As used in this specification, the term “framework type” is used in the sense described in the “Atlas of Zeolite Framework Types,” 2001.

As used herein, the numbering scheme for the Periodic Table Groups is used as in Chemical and Engineering News, 63(5), 27 (1985).

The term “wppm” as used herein is defined as parts per million by weight.

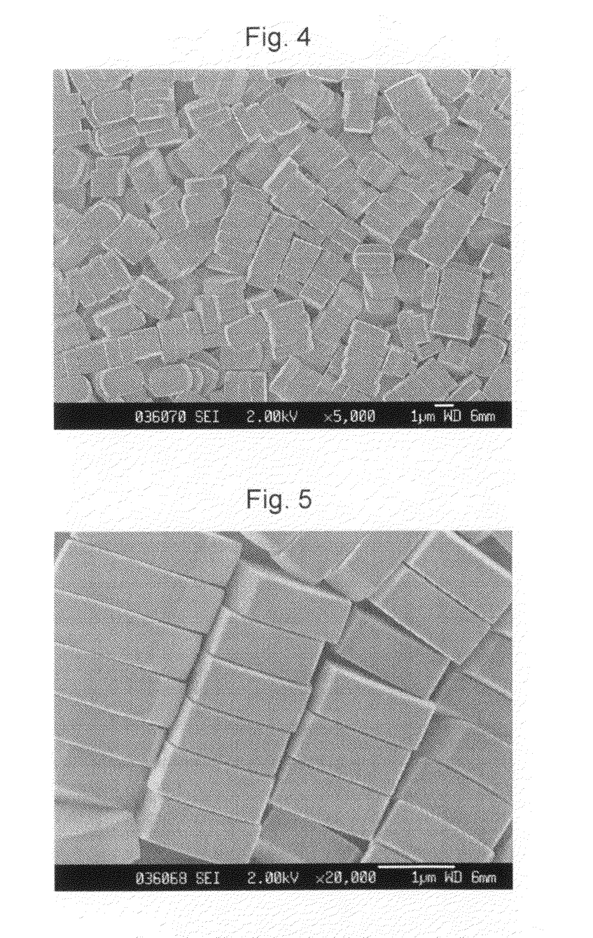

The crystal morphology and uniformity of crystal morphology can be measured by Scanning Electron Microscopy (SEM). A SEM picture having at least 50 visible crystals is used for this purpose. For example, in the case of FIG. 4, it is observed that at least 98% of the crystals have quasi parallelepiped morphology, the rest of the crystals being mainly twinned crystals. In this example, the uniformity of crystal morphology is 98%. The term “substantially uniform quasi parallelepiped morphology” as used herein means that more than 80% of the crystals in a SEM picture having at least 50 visible crystals have quasi parallelepiped morphology.

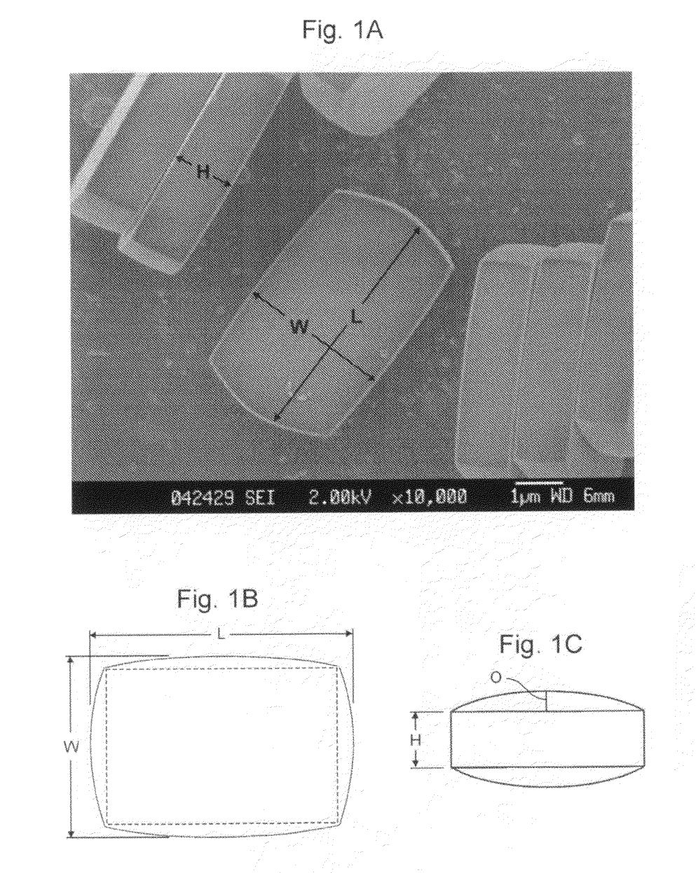

The term “quasi parallelepiped morphology” (QPM) as used herein means a parallelepiped morphology having gently round-off ends. An example of an individual crystal having quasi parallelepiped morphology is illustrated in FIG. 1a. The length (L) of the crystal is the longest dimension of the quasi parallelepiped particle as illustrated in FIGS. 1a and 1b. The width (W) of the crystal is the second longest dimension of the quasi parallelepiped particle as illustrated in FIGS. 1a and 1b. And the height (H) of the crystal is the shortest dimension of the quasi parallelepiped particle as illustrated in FIGS. 1a and 1b. The quasi parallelepiped morphology is further characterized by sharp edges of the crystal particle as shown in FIG. 4.

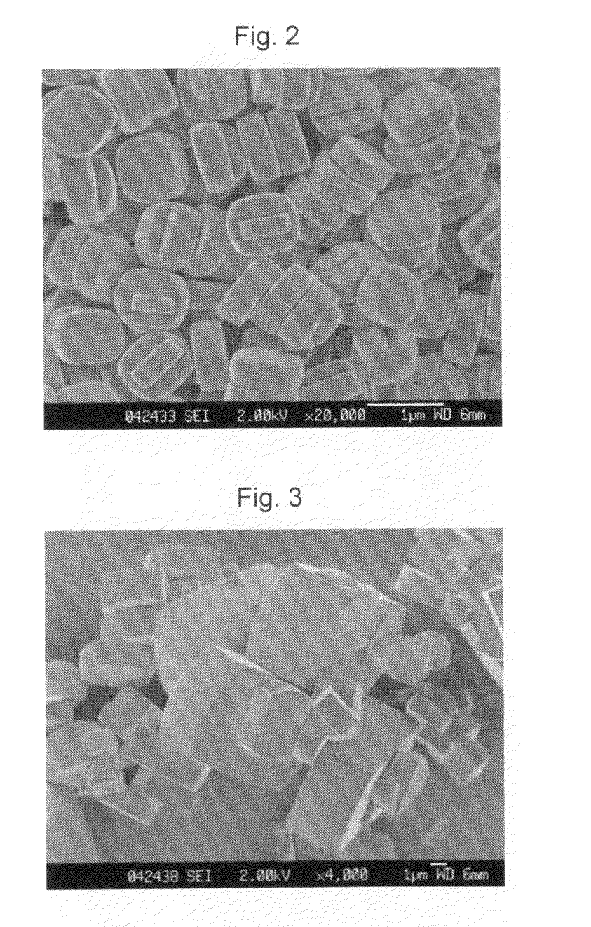

The quasi parallelepiped morphology is yet further characterized by relative smooth and relative flat of the surfaces of the crystal particles as shown in FIG. 4 as comparing to the non-even and/or less smooth surface of the crystal particles as disclosed by researchers at Sogang University, Korea (J. Am. Chem. Soc., 2001, 123, 9769, hereinafter the JACS paper) as shown in FIGS. 1, 3, 4 and 6 of the JACS paper.

The flatness of the crystal surface is defined as the ratio of the offset (O as shown in FIG. 1c from a flat surface over the average of the length and the width, i.e., (L+W)/2, of the crystal. The crystal surface of the crystalline molecular sieve of this disclosure is substantially flat. The flatness of the crystal surface used in this disclosure is measured by SEM with at least 50 visible crystals per SEM picture. The term “substantial flat” as used herein means a flatness of the largest crystal surface of less than 0.05.

The crystal size and uniformity of crystal size can be measured by Scanning Electron Microscopy (SEM) and/or by laser scattering. When the crystal size and uniformity of crystal size is measured by SEM, a SEM picture having at least 50 visible crystals is used. For example, in the case of FIG. 4, it is observed that at least 95% of the crystals have dimensions (length L, width W, height H) equal to 2.20 (±0.05), 1.40 (±0.05), and 0.65 (±0.05) micrometer, respectively. The numbers between brackets represent the combination of measured variations in crystal size and experimental accuracy inherent to crystal size measurement based on SEM. The uniformity of crystal size when measured by SEM is expressed as a±b %, which means that at least a % of the crystals in a SEM picture having at least 50 visible crystals have a dimension L (the longest dimension) within ±b % of the average dimension L. For example, the uniformity of the crystals of FIG. 4 is 95%±2%. In a case when the uniformity of crystal size is measured by SEM, the term “substantially uniform in size” as used herein means 90%±10%, i.e., at least 90% of the crystals in a SEM picture having at least 50 visible crystals have a dimension L (the longest dimension) within ±10% of the average dimension L.

The ratios of the three dimensions, e.g., length/width (L/W) and length/height (L/H), are calculated from the crystal dimensions measured by SEM. The ratios are calculated based on the average L, W, and H dimensions of the crystals of a specific sample. For example, in the case of FIG. 4, the L/W and L/H ratios are 1.6 and 3.4, respectively.

When the average crystal size and crystal size distribution is measured by laser scattering, Gaussian-type particle size distribution curves are obtained. The average crystal size is expressed by D(50) in micrometer. The crystal size distribution and uniformity are expressed in D(10), D(50), D(90) and span. A D(c) number of, e.g., 1 micrometer, means that c % of the volume of the particles is smaller than 1 micrometer. The span is calculated as [D(90)−D(10)]/D(50) and indicates the width of the particle size distribution. In a case when the uniformity of crystal size is measured by laser scattering, the term “substantially uniform in size” as used herein means a span of 3 or less.

The term “substantially free of crystal twinning” as used herein means that less than 20% of the crystals in a SEM picture having at least 50 visible crystals are twinned crystals. A twinned crystal is defined as a crystal having 90-degree rotational intergrowth.

In some embodiments, the disclosure relates to a crystalline molecular sieve composition of matter having MFI topology, wherein the crystalline molecular sieve composition of matter comprises substantially uniform sized crystals having substantially uniform quasi parallelepiped morphology. In other embodiments, the crystalline molecular sieve composition of matter of this disclosure comprises substantially uniform sized crystals having substantially uniform quasi parallelepiped morphology and substantially free of crystal twinning. In some embodiments, the disclosure relates to a crystalline molecular sieve composition of matter having MFI topology, wherein the crystalline molecular sieve composition of matter comprises substantially uniform sized crystals having substantially uniform quasi parallelepiped morphology and a flatness of less than 0.05 measured by SEM. In other embodiments, the disclosure relates to a crystalline molecular sieve composition of matter having MFI topology, wherein the crystalline molecular sieve composition of matter comprises substantially uniform sized crystals having substantially uniform quasi parallelepiped morphology, substantially free of crystal twinning, and a flatness of less than 0.05 measured by SEM. In yet other embodiments, the crystalline molecular sieve composition of matter of this disclosure has a uniformity of crystal size of 90%±5% when measured by SEM or a span of less than 3 measured by laser scattering.

In some aspects, the crystalline molecular sieve composition of matter has an average molecular sieve crystal size of at least 1 micrometer, preferably at least 2 micrometer measured by SEM or laser scattering. In other aspects, the crystalline molecular sieve composition of matter has an average length/width ratio of the molecular sieve crystal in the range of 1 to 10 and a length/height ratio of the molecular sieve crystal in the range of 2 to 20.

In a preferred embodiment, the crystalline molecular sieve composition of matter is an aluminosilicate ZSM-5 zeolite. In another preferred embodiment, the crystalline molecular sieve composition of matter is silicalite-1.

In some embodiments, the uniformity of crystal size (measured by SEM) of the crystalline molecular sieve composition of matter is 90%±10%, 95%±10%, or 98%±10%, preferably 90%±5%, 95%±5%, or 98%±5%, more preferably 90%±2% or 95%±2%, and most preferably 98%±2%.

In other embodiments of this disclosure, the uniformity of crystal size (measured by laser scattering) of the crystalline molecular sieve composition of matter has a span of 3 or less, preferably 2 or less, more preferably 1.5 or less, even more preferably 1.3 or less, yet even more preferably 1.2 or less, yet even more preferably 1.1 or less, yet even more preferably 1.0 or less, and most preferably 0.9 or less.

In some embodiments of this disclosure, the uniformity of the quasi parallelepiped morphology of the crystalline molecular sieve composition of matter of this disclosure is at least 90%, preferably at least 95%, more preferably at least 97%, and most preferably at least 98%, based on a SEM picture with at least 50 visible crystals.

In some embodiments of this disclosure, the crystalline molecular sieve composition of matter of this disclosure has less than 10%, preferably less than 5%, more preferably less than 3%, even more preferably less than 2%, and most preferably less than 1% of crystal twinning, based on a SEM picture with at least 50 visible crystals.

In some embodiments of this disclosure, the crystalline molecular sieve composition of matter of this disclosure has an average flatness of the crystal surface measured using the crystal surface having the largest surface area of each individual crystal particle (based on a SEM picture with at least 50 visible crystals) of less than 0.05, preferably less than 0.04, more preferably less than 0.03, even more preferably less than 0.02, and most preferably less than 0.01.

### Formulation of the Hydrothermal Reaction Mixtures

Synthetic molecular sieves are often prepared from aqueous hydrothermal reaction mixtures (synthesis mixture(s) or synthetic gel(s)) comprising sources of appropriate oxides. Organic directing agents may also be included in the hydrothermal reaction mixture for the purpose of influencing the production of a molecular sieve having the desired structure. The use of such directing agents is discussed in an article by Lok et al. entitled “The Role of Organic Molecules in Molecular Sieve Synthesis” appearing in Zeolites, Vol. 3, October, 1983, pp. 282-291.

After the components of the hydrothermal reaction mixture are properly mixed with one another, the hydrothermal reaction mixture is subjected to appropriate crystallization conditions. Such conditions usually involve heating of the hydrothermal reaction mixture to an elevated temperature possibly with stirring. Room temperature aging of the hydrothermal reaction mixture is also desirable in some instances.

After the crystallization of the hydrothermal reaction mixture is complete, the crystalline product may be recovered from the remainder of the hydrothermal reaction mixture, especially the liquid contents thereof. Such recovery may involve filtering the crystals and washing these crystals with water. However, in order to remove the entire undesired residue of the hydrothermal reaction mixture from the crystals, it is often necessary to subject the crystals to a high temperature calcination e.g., at 500° C., possibly in the presence of oxygen. Such a calcination treatment not only removes water from the crystals, but this treatment also serves to decompose and/or oxidize the residue of the organic directing agent which may be occluded in the pores of the crystals, possibly occupying ion exchange sites therein.

The crystalline molecular sieve composition of matter of this disclosure may be prepared from a hydrothermal reaction mixture comprising at least one source of at least one tetravalent element (Y), at least one source of at least one trivalent element (X), at least one source of hydroxide ion, at least one directing-agent (R), water, and optionally sources of alkali or alkali earth metal (M), e.g., sodium, or potassium, and water, the hydrothermal reaction mixture having a composition, in terms of mole ratios, within the following ranges as shown in Table 1:

TABLE 1

| Reactants | Useful | Preferred | More Preferred |
|---|---|---|---|
| Y/X₂ | 10-infinity | 50-10000 | 100-1000 |
| H₂O/YO₂ | 10-1000 | 15-500 | 20-200 |
| OH⁻/YO₂* | 0.01-5 | 0.03-2 | 0.05-1 |
| M/YO₂ | 0-2 | 0-1 | 0-0.5 |
| R/YO₂ | 0.075-5 | 0.1-2 | 0.1-1 |

*The OH⁻/YO₂ is calculated without correction of trivalent element source.

The sources of the various elements required in the final product may be any of those in commercial use or described in the literature, as may the method of preparation of the synthesis mixture.

Y is a tetravalent element selected from Groups 4-14 of the Periodic Table of the Elements, such as silicon and/or germanium, preferably silicon. In some embodiments of this disclosure, the source of Y comprises solid YO₂, preferably about 30 wt % solid YO₂ in order to obtain the crystal product of this disclosure. When YO₂ is silica, the use of a silica source containing preferably about 30 wt % solid silica, e.g., silica sold by Degussa under the trade names Aerosil® or Ultrasil (a precipitated, spray dried silica containing about 90 wt % silica), an aqueous colloidal suspension of silica, for example one sold by Grace Davison under the trade name Ludox®, or HiSil® (a precipitated hydrated SiO₂ containing about 87 wt % silica, about 6 wt % free H₂O and about 4.5 wt % bound H₂O of hydration and having a particle size of about 0.02 micrometer) favors crystal formation from the above mixture. Preferably, therefore, the YO₂, e.g., silica, source contains about 30 wt % solid YO₂, e.g., silica, and more preferably about 40 wt % solid YO₂, e.g., silica. The source of silicon may also be a silicate, e.g., an alkali metal silicate, or a tetraalkylorthosilicate.

X is a trivalent element selected from Groups 3-13 of the Periodic Table of the Elements, such as aluminum, and/or boron, and/or iron and/or gallium, preferably aluminum. The source of X₂, e.g., aluminum, is preferably aluminum sulfate or hydrated alumina. Other aluminum sources include, for example, other water-soluble aluminum salts, sodium aluminate, or an alkoxide, e.g., aluminum isopropoxide, or aluminum metal, e.g., in the form of chips.

The alkali or alkali earth metal element is advantageously lithium, sodium, potassium, calcium, or magnesium. The source of alkali or alkali earth metal element is advantageously being metal oxide, metal chloride, metal fluoride, metal sulfate, metal nitrate, or metal aluminate. The sodium source advantageously being sodium hydroxide or sodium aluminate. The alkali metal may also be replaced by ammonium (NH₄⁺) or its equivalents, e.g., alkyl-ammonium ion.

Directing agent R comprises at least one of tetrapropylammonium salts, such as, tetrapropylammonium hydroxide (TPAOH), tetrapropylammonium chloride (TPACl), tetrapropylammonium bromide (TPABr), tetrapropylammonium iodide (TPAI), and tetrapropylammonium fluoride (TPAF).

The source of OH⁻ source is advantageously organic ammonium hydroxide, such as TPAOH, ammonium hydroxide, alkali metal oxide, e.g., Li₂O, Na₂O, K₂O, Rb₂O, Cs₂O, Fr₂O, or any combination thereof; alkali metal hydroxide, e.g., LiOH, NaOH, KOH, RbOH, CsOH, FrOH, or any combination thereof; ammonium hydroxide, alkali earth metal oxide, e.g., BeO, MgO, CaO, SrO, BaO, RaO, or any combination thereof; alkali earth metal hydroxide, e.g., Be(OH)₂, Mg(OH)₂, Ca(OH)₂, Sr(OH)₂, Ba(OH)₂, Ra(OH)₂, or any combination thereof; oxide(s) or hydroxide(s) of any element selected from Groups 3-17; and any combination thereof.

The OH⁻/Y, e.g., OH⁻/Si molar ratio as used in this disclosure does not include correction of acid in the hydrothermal reaction mixture. It is calculated based on the total mole of hydroxide added to the hydrothermal reaction mixture.

In some embodiments of this disclosure, the OH⁻/Y molar ratio as used in this disclosure is in the range of from 0.01 to 5 and the R/Y, e.g., R/Si molar ratio as used in this disclosure is in the range of from 0.075 to 5. The following OH⁻/Y molar ratios are useful lower OH⁻/Y molar ratios limits: 0.01, 0.05, 0.1, 0.2, 0.3, 0.4, and 0.5. The following OH⁻/Y molar ratios are useful upper OH⁻/Y molar ratios limits: 5, 2, 1.5, 1, 0.5, 0.4, 0.3, and 0.2. The OH⁻/Y molar ratio of a synthesis mixture useful for this disclosure ideally falls in a range between any one of the above-mentioned lower limits and any one of the above-mentioned upper limits, so long as the lower limit is less than or equal to the upper limit. The following R/Y molar ratios are useful lower R/Y molar ratios limits: 0.075, 0.08, 0.09, 0.1, 0.2, 0.3, 0.4, 0.5, and 1. The following R/Y molar ratios are useful upper R/Y molar ratios limits: 5, 2, 1.5, 1, 0.5, 0.4, 0.3, and 0.2. The R/Y molar ratio of a synthesis mixture useful for this disclosure ideally falls in a range between any one of the above-mentioned lower limits and any one of the above-mentioned upper limits, so long as the lower limit is less than or equal to the upper limit.

In some embodiments of this disclosure, the M/Y, e.g., M/Si molar ratio as used in this disclosure is in the range of from 0 to 2. In other embodiments, the M/Y molar ratio as used in this disclosure is in the range of from 0 to 1. In yet other embodiments, the M/Y molar ratio as used in this disclosure is in the range of from 0 to 0.5. The following M/Y molar ratios are useful lower M/Y molar ratios limits: 0, 0.001, 0.005, 0.01, 0.05, 0.1, 0.2, 0.3, 0.4, 0.5, and 1. The following M/Y molar ratios are useful upper M/Y molar ratios limits: 2, 1.5, 1, 0.5, 0.4, 0.3, and 0.2. The M/Y molar ratio of a synthesis mixture useful for this disclosure ideally falls in a range between any one of the above-mentioned lower limits and any one of the above-mentioned upper limits, so long as the lower limit is less than or equal to the upper limit.

In some embodiments of this disclosure, the H₂O/Y, e.g., H₂O/Si molar ratio as used in this disclosure is in the range of from 10 to 1000. In other embodiments, the H₂O/Y molar ratio as used in this disclosure is in the range of from 15 to 500. In yet other embodiments, the H₂O/Y molar ratio as used in this disclosure is in the range of from 20 to 200. The following H₂O /Y molar ratios are useful lower H₂O/Y molar ratios limits: 10, 15, 20, 25, 30, 50, 100, 200, and 500. The following H₂O/Y molar ratios are useful upper H₂O/Y molar ratios limits: 1000, 800, 600, 500, 400, 300, 200, 100, 90, 80, 70, 60, and 50. The H₂O/Y molar ratio of a synthesis mixture useful for this disclosure ideally falls in a range between any one of the above-mentioned lower limits and any one of the above-mentioned upper limits, so long as the lower limit is less than or equal to the upper limit.

In some embodiments of this disclosure, the Y/X₂, e.g., Si/Al₂, molar ratio as used in this disclosure is in the range of from 10 to infinity. In other embodiments, the Y/X₂ molar ratio as used in this disclosure is in the range of from 50 to 10000. In yet other embodiments, the Y/X₂ molar ratio as used in this disclosure is in the range of from 100 to 1000. The following Y/X₂ molar ratios are useful lower Y/X₂ molar ratios limits: 10, 50, 100, 200, 300, 400, and 500. The following Y/X₂ molar ratios are useful upper Y/X₂ molar ratios limits: infinity, 10000, 5000, 4000, 3000, 2000, 1000, 900, 800, 700, 600, 500, 400, 300, 200, 100, and 50. The Y/X₂ molar ratio of a synthesis mixture useful for this disclosure ideally falls in a range between any one of the above-mentioned lower limits and any one of the above-mentioned upper limits, so long as the lower limit is less than or equal to the upper limit.

It should be realized that the hydrothermal reaction mixture components can be supplied by more than one source. The hydrothermal reaction mixture can be prepared either batchwise or continuously. Crystal size and crystallization time of the ZSM-5 zeolite of this disclosure may vary with the nature of the hydrothermal reaction mixture employed and the crystallization conditions.

It will be understood by a person skilled in the art that the synthesis mixture having a composition within the molar ranges as discussed above means that the synthesis mixture is the product of mixing, adding, reacting, or by any means of providing such a mixture, wherein such product has a composition within the molar ranges as discussed above. The product of mixing, adding, reacting, or by any means of providing such a mixture may or may not contain individual ingredients when the synthesis mixture was prepared. The product of mixing, adding, reacting, or by any means of providing such a mixture, may even contain reaction product of individual ingredients when the synthesis mixture was prepared by mixing, adding, reacting, or by any means of providing such a mixture.

Optionally the hydrothermal reaction mixture may contain seed crystals. It is well known that seeding a molecular sieve synthesis mixture frequently has beneficial effects, for example in controlling the particle size of the product, avoiding the need for an organic template, accelerating synthesis, and improving the proportion of product that is of the intended framework type. In some embodiments of this disclosure, the synthesis of the crystalline molecular sieve is facilitated by the presence of 0 to about 25 wt %, preferably about 1 to about 5 wt %, seed crystals based on total weight of tetrahedral element oxide (e.g., silica) of the hydrothermal reaction mixture.

### Crystallization Conditions

The hydrothermal reaction of this disclosure is carried with agitation. The rate of the agitation is measured by the rotation speed of the stirrer in rotation per minute (RPM), by the tip-speed in m/s, or by volume average stirring speed. If a stirrer having a diameter of m (meter) is rotated with n RPM, then the tip speed is calculated as π*m*n/60 (m/s). In the vessels used in this disclosure, a stirring rate of 100 RPM corresponds to a tip speed of 0.146 m/s. The effect of stirring may also expressed as volume average speed which is calculated as π*m*n/180 (m/s).

The mixture is crystallized under crystallization conditions comprising a temperature in the range of from 125° C. to 250° C., preferably 130° C. to 200° C., a crystallization time from about 1 hour to 1000 hours, preferably from about 5 hours to about 200 hours, more preferably from about 10 hours to about 100 hours; a heating rate in the range from at least 10° C./h, preferably at least 20° C./h, and most preferably at least 50° C./h, and a stirring speed at least 5 RPM, preferably at least 10 RPM, more preferably at least 50 RPM, even more preferably at least 100 RPM, yet even more preferably at least 150 RPM, still yet more preferably at least 200 RPM, still yet more preferably at least 250 RPM, still yet more preferably at least 300 RPM, and most preferably 350 RPM, or a stirring speed at least 0.005 m/s, preferably at least 0.01 m/s, more preferably at least 0.05 m/s, even more preferably at least 0.1 m/s, yet even more preferably at least 0.15 m/s, still yet more preferably at least 0.2 m/s, still yet more preferably at least 0.25 m/s, still yet more preferably at least 0.5 m/s, and most preferably 1 m/s, or a volume average stirring speed at least 0.0016 m/s, preferably at least 0.0033 m/s, more preferably at least 0.016 m/s, even more preferably at least 0.033 m/s, yet even more preferably at least 0.05 m/s, still yet more preferably at least 0.067 m/s, still yet more preferably at least 0.0833 m/s, still yet more preferably at least 0.167 m/s, and most preferably 0.333 m/s.

In some embodiments, the crystallization conditions comprises a combination of a heating rate at least 10° C./h and a stirring speed at least 250 RPM or a tip speed at least 0.146 m/s. In other embodiments, the crystallization conditions comprises a combination of a heating rate at least 50° C./h and a stirring speed at least 100 RPM or a tip speed at least 0.100 m/s. In yet other embodiments, the crystallization conditions comprises a combination of a heating rate at least 100° C./h and a stirring speed at least 50 RPM or a tip speed at least 0.050 m/s.

All heating rate as used herein means heating a mixture continuously for at least 5 minutes, preferably at least 10 minutes, at the specified heating rate. All stirring speed as used herein means stirring a mixture continuously for at least 5 minutes, preferably at least 10 minutes, at the specified stirring speed.

The procedure may optionally include an aging period, either at room temperature (˜25° C.) or at a moderately elevated temperature (less than 100° C.), with or without agitation, before the hydrothermal treatment (“hydrothermal reaction”) at more elevated temperature. The aging period may last from 0 to 500 hours, preferably from 1 to 100 hours, more preferably from 2 to 50 hours, and most preferably from 5 to 20 hours.

The crystalline molecular sieve from the synthesis may further be filtrated, washed with water, and/or dried. The crystalline molecular sieve of this disclosure formed by crystallization may be recovered and subjected for further treatment, such as, ion-exchange with ammonium salt(s) (e.g., ammonium hydroxide, ammonium nitrate, ammonium chloride, ammonium sulfate, ammonium phosphate, or any combination thereof), impregnation, dealumination, and/or calcination in an oxidative atmosphere (e.g., air, gas with an oxygen partial pressure of greater than 0 kPa-a) at a temperature of greater than 200° C., preferably at least 300° C., more preferably at least 400° C., and most preferably at least 500° C.

Impregnation allows deposition of metal salts on the molecular sieve, e.g. salts of noble metals. Dealumination can be done, e.g., by steaming or by any chemical treatment. These treatments result in modifying the molecular sieve framework composition.

The molecular sieve may be bound with a matrix material such as clay or silica to increase the physical strength of the material for its use as a catalyst in a variety of processes.

### INDUSTRIAL APPLICATIONS

The crystalline molecular sieve of this disclosure is useful in the production and conversion of organic compounds, for example cracking, hydrocracking, dewaxing, isomerization (including e.g. olefin summarization and skeletal summarization e.g. of butane), oligomerization, trimerization, polymerization, alkylation, dealkylation, hydrogenation, dehydrogenation, dehydration, cyclization and aromatization. The present invention therefore provides a process for the production or conversion of an organic compound comprising the use of a catalyst of the molecular sieve described above. The molecular sieve can also be used (either as initially prepared or in a modified form) in a selective adsorption process e.g. a separation or purification.

These and other facets of the present invention are exemplified by the following Examples.

### Examples

In the Examples, the XRD diffraction patterns of the as-synthesized materials were recorded on a STOE Stadi-P Combi transmission XRD using copper Kα radiation in the 2θ range of 2 to 40 degrees.

The SEM images were obtained on a JEOL JSM-6340F Field-Emission-Gun scanning electron microscope is used, operating at 2 kV and 12 μA. The crystal size was measured by averaging the size of multiple crystals as shown in the SEM.

The particle size analysis is performed using a Mastersizer APA2000, from Malvern Instruments Limited, equipped with a 4 mW laser beam, based on laser scattering by randomly moving particles in a liquid medium. The samples to be measured are dispersed in water and sonicated in situ to ensure proper dispersion.

Autoclaves with a capacity of 30 mL were used for the synthesis. These autoclaves were equipped with heating jacket, internal thermocouples, and mechanical stir.

The following Table 2 lists all chemicals, raw materials, and their sources used in the Examples of this disclosure.

TABLE 2

| Name | Composition | Source |
|---|---|---|
| Tetrapropylammonium | 20 wt % in water | Aldrich |
| Hydroxide (TPAOH) | | |
| Tetraethylorthosilicate (TEOS) | 98% | Aldrich |
| NaOH | 98% | Aldrich |
| Al(OH)₃ | 96+% | Aldrich |
| Aluminum Sulfate | 98+% | Aldrich |

### Examples 1-24

Examples 1-24 were prepared with the following procedure:
(a) aqueous TPAOH was added to TEOS under stirring;
(b) the mixture of step (a) was stirred for 1 hour at 25° C.;
(c) an aqueous solution composed of 10 wt % NaOH and 7.8 wt % Al(OH)₃ was added slowly under stirring;
(d) water was added to the mixture of step (c);
(e) the mixture of step (d) was stirred at 25° C. for 12 hours; then
(f) the mixture of step (e) was transferred to a stirred autoclave;
(g) the autoclave was heated to 160° C. with different heating rates;
(h) the autoclave was maintained at 160° C. for 40 hours with different stirring rates;
(i) the autoclave was cooled down to 25° C. and the crystals were recovered and washed using centrifugation or filtration, and dried at 60° C. for 24 hours; and
(j) the product of step (i) was analyzed by XRD, laser scatting, and SEM.

The following Tables 3-5 summarized the slurry composition, crystallization conditions, and product characterization results, such as quasi parallelepiped morphology (QPM) and hexagonal morphology (HEX).

TABLE 3

| | Example | | | | | | | |
|---|---|---|---|---|---|---|---|---|
| | 1 | 2 | 3 | 4 | 5 | 6 | 7 | 8 |
| Al/Si (molar) | 0 | 0 | 0 | 0.004 | 0.004 | 0.004 | 0.02 | 0.02 |
| TPA/Si (molar) | 0.125 | 0.125 | 0.125 | 0.125 | 0.125 | 0.125 | 0.125 | 0.125 |
| H₂O/Si (molar) | 50 | 50 | 50 | 50 | 50 | 50 | 50 | 50 |
| M/Si (molar) | 0 | 0 | 0 | 0.01 | 0.01 | 0.01 | 0.05 | 0.05 |
| OH⁻/Si (molar) | 0.125 | 0.125 | 0.125 | 0.135 | 0.135 | 0.135 | 0.175 | 0.175 |
| Heating rate | 10 | 10 | 10 | 10 | 10 | 10 | 10 | 10 |
| (° C./h) | | | | | | | | |
| Stirring rate | 50 | 150 | 350 | 50 | 150 | 350 | 50 | 150 |
| (RPM) | | | | | | | | |
| XRD | MFI | MFI | MFI | MFI | MFI | MFI | MFI | MFI |
| D50 (micron) | 1.4 | 1.0 | 1.7 | 0.6 | 1.0 | 1.2 | 12.3 | 1.4 |
| Span | 4.7 | 2.7 | 2.3 | 7.4 | 2.1 | 2.2 | 2.4 | 1.4 |
| QPM uniformity | 80% | 80% | 90% | 50% | 80% | 80% | | 90% |
| Twinning | 20% | 20% | 10% | 50% | 20% | 20% | | 10% |

TABLE 4

| | Example | | | | | | | |
|---|---|---|---|---|---|---|---|---|
| | 9 | 10 | 11 | 12 | 13 | 14 | 15 | 16 |
| Al/Si (molar) | 0.02 | 0.1 | 0.1 | 0.1 | 0 | 0 | 0 | 0.004 |
| TPA/Si (molar) | 0.125 | 0.125 | 0.125 | 0.125 | 0.125 | 0.125 | 0.125 | 0.125 |
| H₂O/Si (molar) | 50 | 50 | 50 | 50 | 50 | 50 | 50 | 50 |
| M/Si (molar) | 0.05 | 0.25 | 0.25 | 0.25 | 0 | 0 | 0 | 0.01 |
| OH⁻/Si (molar) | 0.175 | 0.375 | 0.375 | 0.375 | 0.125 | 0.125 | 0.125 | 0.135 |
| Heating rate (° C./h) | 10 | 10 | 10 | 10 | 50 | 50 | 50 | 50 |
| Stirring rate | 350 | 50 | 150 | 350 | 50 | 150 | 350 | 50 |
| (RPM) | | | | | | | | |
| XRD | MFI | dense | dense | dense | MFI | MFI | MFI | MFI |
| D50 (micron) | 1.1 | 17.5 | 20.7 | 17.5 | 4.3 | 1.1 | 2.0 | 1.1 |
| Span | 2.0 | 2.9 | 2.2 | 2.0 | 2.2 | 1.9 | 1.1 | 2.1 |
| QPM uniformity | 80% | | | | 50% | 95% | 95% | 90% |
| Twinning | 20% | | | | 50% | 5% | 5% | 10% |
| L (micron) | | | | | | 1.10 | | |
| W (micron) | | | | | | 0.80 | | |
| H (micron) | | | | | | 0.35 | | |
| L/W/H accuracy | | | | | | 0.10 | | |
| (micron) | | | | | | | | |
| Size uniformity at | | | | | | 9% | | |
| 95% ± % | | | | | | | | |
| L/W | | | | | | 1.4 | | |
| L/H | | | | | | 3.1 | | |

TABLE 5

| | Example | | | | | | | |
|---|---|---|---|---|---|---|---|---|
| | 17 | 18 | 19 | 20 | 21 | 22 | 23 | 24 |
| Al/Si (molar) | 0.004 | 0.004 | 0.02 | 0.02 | 0.02 | 0.1 | 0.1 | 0.1 |
| TPA/Si (molar) | 0.125 | 0.125 | 0.125 | 0.125 | 0.125 | 0.125 | 0.125 | 0.125 |
| H₂O/Si (molar) | 50 | 50 | 50 | 50 | 50 | 50 | 50 | 50 |
| M/Si (molar) | 0.01 | 0.01 | 0.05 | 0.05 | 0.05 | 0.25 | 0.25 | 0.25 |
| OH⁻/Si (molar) | 0.135 | 0.135 | 0.175 | 0.175 | 0.175 | 0.375 | 0.375 | 0.375 |
| Heating rate(° C./h) | 50 | 50 | 50 | 50 | 50 | 50 | 50 | 50 |
| Stirring rate (RPM) | 150 | 350 | 50 | 150 | 350 | 50 | 150 | 350 |
| XRD | MFI | MFI | MFI | MFI | MFI | dense | dense | dense |
| D50 (micron) | 1.3 | 1.5 | 11.7 | 4.1 | 1.8 | 21.5 | 17.0 | 19.5 |
| Span | 2.1 | 0.8 | 4.1 | 2.2 | 1.3 | 26.4 | 2.9 | 2.2 |
| QPM Uniformity | 95% | 98% | | 80% | 90% | | | |
| Twinning | 5% | 2% | | 20% | 10% | | | |
| L (micron) | 1.10 | 2.20 | | | | | | |
| W (micron) | 0.80 | 1.40 | | | | | | |
| H (micron) | 0.35 | 0.65 | | | | | | |
| L/W/H accuracy | 0.10 | 0.05 | | | | | | |
| (micron) | | | | | | | | |
| Size uniformity at | 9% | 2% | | | | | | |
| 95% ± % | | | | | | | | |
| L/W | 1.4 | 1.6 | | | | | | |
| L/H | 3.1 | 3.4 | | | | | | |

### Examples 25-36

Examples 25-36 were prepared according the following sequence of addition: first aqueous TPAOH, then aqueous NaOH, then TEOS, then an aqueous solution composed of 10 wt % NaOH and 7.8 wt % Al(OH)₃, then water to form a mixture with the composition of the following table. Then the mixture was stirred at 25° C. for 4 hours; then the mixture was transferred to a stirred autoclave; then the autoclave was heated to 160° C. with 100° C./h heating rate; then the autoclave was maintained at 160° C. for 40 hours with 350 RPM stirring rate; then the autoclave was cooled down to 25° C. and the crystals were recovered and washed using centrifugation or filtration, and dried at 60° C. for 24 hours; then the product was analyzed by XRD, laser scatting, and SEM.

The following Tables 6-7 summarized the slurry composition, crystallization conditions, and product characterization results, such as quasi parallelepiped morphology (QPM) and hexagonal morphology (HEX).

TABLE 6

| | Example | | | | |
|---|---|---|---|---|---|
| | 25 | 26 | 27 | 28 | 29 |
| Al/Si (molar) | 0.0033 | 0.0033 | 0.0033 | 0.0033 | 0.0033 |
| TPA/Si (molar) | 0.125 | 0.1 | 0.075 | 0.125 | 0.1 |
| H₂O/Si (molar) | 50 | 50 | 50 | 50 | 50 |
| M/Si (molar) | 0.00825 | 0.00825 | 0.025 | 0.025 | 0.025 |
| OH⁻/Si (molar) | 0.13325 | 0.10825 | 0.1 | 0.15 | 0.125 |
| Heating rate (° C./h) | 100 | 100 | 100 | 100 | 100 |
| Stirring rate (RPM) | 350 | 350 | 350 | 350 | 350 |
| XRD | MFI | MFI | MFI | MFI | MFI |
| D50 (micron) | 2.2 | 4.5 | 11.8 | 2.3 | 3.9 |
| Span | 0.9 | 1.0 | 1.4 | 1.0 | 0.8 |
| QPM uniformity | 98% | 95% | 50% | 98% | 98% |
| Twinning | 2% | 5% | 20% | 2% | 2% |
| L (micron) | 2.90 | 7.10 | | 2.50 | 6.00 |
| W (micron) | 1.70 | 3.80 | | 1.60 | 3.60 |
| H (micron) | 0.80 | 1.30 | | 0.80 | 1.40 |
| L/W/H accuracy | 0.10 | 0.20 | | 0.10 | 0.10 |
| (micron) | | | | | |
| Size uniformity at | 3% | 3% | | 4% | 2% |
| 95% ± % | | | | | |
| L/W | 1.7 | 1.9 | | 1.6 | 1.7 |
| L/H | 3.6 | 5.5 | | 3.1 | 4.3 |

TABLE 7

| | Example | | | | | | |
|---|---|---|---|---|---|---|---|
| | 30 | 31 | 32 | 33 | 34 | 35 | 36 |
| Al/Si (molar) | 0.0033 | 0.0033 | 0.0033 | 0.0033 | 0.0033 | 0.0033 | 0.0033 |
| TPA/Si (molar) | 0.075 | 0.05 | 0.125 | 0.1 | 0.075 | 0.05 | 0.05 |
| H₂O/Si (molar) | 50 | 50 | 50 | 50 | 50 | 50 | 50 |
| M/Si (molar) | 0.05 | 0.05 | 0.05 | 0.05 | 0.075 | 0.075 | 0.1 |
| OH⁻/Si (molar) | 0.125 | 0.1 | 0.175 | 0.15 | 0.15 | 0.125 | 0.15 |
| Heating rate (° C./h) | 100 | 100 | 100 | 100 | 100 | 100 | 100 |
| Stirring rate (RPM) | 350 | 350 | 350 | 350 | 350 | 350 | 350 |
| XRD | MFI | MFI | MFI | MFI | MFI | MFI | MFI |
| D50 (micron) | 7.9 | 11.6 | 1.8 | 3.7 | 6.0 | 5.7 | 6.3 |
| Span | 1.4 | 2.4 | 0.8 | 1.1 | 1.2 | 1.2 | 1.7 |
| QPM uniformity | 95% | | 98% | 95% | 95% | | |
| Twinning | 2% | | 2% | 2% | 2% | | |
| L (micron) | | | 2.20 | 4.70 | 7.20 | | |
| W (micron) | | | 1.60 | 2.80 | 4.90 | | |
| H (micron) | | | 0.80 | 1.30 | 2.30 | | |
| L/W/H accuracy | | | 0.10 | 0.10 | 0.20 | | |
| (micron) | | | | | | | |
| Size uniformity at | | | 5% | 2% | 3% | | |
| 95% ± % | | | | | | | |
| L/W | | | 1.4 | 1.7 | 1.5 | | |
| L/H | | | 2.8 | 3.6 | 3.1 | | |

### Examples 37-54

Examples 37-54 were prepared with the following procedure:
(a) aqueous TPAOH was added to TEOS under stirring;
(b) the mixture of step (a) was stirred for 1 hour at 25° C.;
(c) an aqueous solution composed of 10 wt % NaOH and 7.8 wt % Al(OH)₃ was added slowly under stirring;
(d) water was added to the mixture of step (c);
(e) the mixture of step (d) was stirred at different temperatures and for different amounts of time; then
(f) the mixture of step (e) was transferred to a stirred autoclave;
(g) the autoclave was heated to different temperatures with different heating rates;
(h) the autoclave was maintained at this temperature for 40 hours with 350 RPM stirring rate;
(i) the autoclave was cooled down to 25° C. and the crystals were recovered and washed using centrifugation or filtration, and dried at 60° C. for 24 hours; and
(j) the product of step (i) was analyzed by XRD, laser scatting, and SEM.

The following Tables 8-10 summarized the slurry composition, crystallization conditions, and product characterization results, such as quasi parallelepiped morphology (QPM) and hexagonal morphology (HEX).

TABLE 8

| | Example | | | | | |
|---|---|---|---|---|---|---|
| | 37 | 38 | 39 | 40 | 41 | 42 |
| Al/Si (molar) | 0.004 | 0.004 | 0.004 | 0.004 | 0.004 | 0.004 |
| TPA/Si (molar) | 0.2 | 0.3 | 0.5 | 0.125 | 0.125 | 0.125 |
| H₂O/Si (molar) | 50 | 50 | 50 | 30 | 40 | 100 |
| M/Si (molar) | 0.01 | 0.01 | 0.01 | 0.01 | 0.01 | 0.01 |
| OH⁻/Si (molar) | 0.21 | 0.31 | 0.51 | 0.135 | 0.135 | 0.135 |
| T of step (e) (° C.) | 25 | 25 | 25 | 25 | 25 | 25 |
| Time of step (e) (h) | 12 | 12 | 12 | 12 | 12 | 12 |
| T of step (g) (° C.) | 160 | 160 | 160 | 160 | 160 | 160 |
| Time of step (h) (h) | 40 | 40 | 40 | 40 | 40 | 40 |
| Heating rate (° C./h) | 100 | 100 | 100 | 100 | 100 | 100 |
| Stirring rate (RPM) | 350 | 350 | 350 | 350 | 350 | 350 |
| XRD | MFI | MFI | MFI | MFI | MFI | MFI |
| D50 (micron) | 0.7 | 0.4 | 0.8 | 0.9 | 1.2 | 1.9 |
| Span | 2.2 | 3.3 | 2.3 | 0.8 | 0.8 | 1.0 |
| QPM uniformity | 95% | 95% | 95% | 90% | 98% | 98% |
| Twinning | 5% | 5% | 5% | 10% | 2% | 2% |
| L (micron) | 0.50 | 0.25 | | 1.10 | 1.70 | 3.30 |
| W (micron) | 0.40 | 0.25 | | 0.90 | 1.20 | 1.90 |
| H (micron) | 0.20 | 0.10 | | 0.45 | 0.50 | 0.70 |
| L/W/H accuracy (micron) | 0.05 | 0.05 | | 0.10 | 0.10 | 0.10 |
| Size uniformity at 95% ± % | 10% | 20% | | 9% | 6% | 3% |
| L/W | 1.3 | 1.0 | | 1.2 | 1.4 | 1.7 |
| L/H | 2.5 | 2.5 | | 2.4 | 3.4 | 4.7 |

TABLE 9

| | Example | | | | | |
|---|---|---|---|---|---|---|
| | 43 | 44 | 45 | 46 | 47 | 48 |
| Al/Si (molar) | 0.004 | 0.004 | 0.004 | 0.004 | 0.004 | 0.004 |
| TPA/Si (molar) | 0.125 | 0.125 | 0.125 | 0.125 | 0.125 | 0.125 |
| H₂O/Si (molar) | 50 | 50 | 50 | 50 | 50 | 50 |
| M/Si (molar) | 0.01 | 0.01 | 0.01 | 0.01 | 0.01 | 0.01 |
| OH⁻/Si (molar) | 0.135 | 0.135 | 0.135 | 0.135 | 0.135 | 0.135 |
| T of step (e) (° C.) | 25 | 25 | 25 | 25 | 25 | 25 |
| Time of step (e) (h) | 12 | 12 | 12 | 12 | 12 | 12 |
| T of step (g) (° C.) | 130 | 150 | 170 | 190 | 160 | 160 |
| Time of step (h) (h) | 40 | 40 | 40 | 40 | 40 | 40 |
| Heating rate (° C./h) | 100 | 100 | 100 | 100 | 5 | 20 |
| Stirring rate (RPM) | 350 | 350 | 350 | 350 | 350 | 350 |
| XRD | MFI | MFI | MFI | MFI | MFI | MFI |
| D50 (micron) | 1.6 | 1.6 | 2.4 | 1.9 | 0.6 | 1.0 |
| Span | 0.7 | 0.8 | 1.2 | 1.0 | 1.0 | 1.2 |
| QPM uniformity | 95% | 95% | 95% | 95% | 90% | 95% |
| Twinning | 5% | 5% | 5% | 5% | 10% | 5% |
| L (micron) | 2.30 | 2.30 | | | 0.80 | 0.90 |
| W (micron) | 1.80 | 1.80 | | | 0.70 | 0.70 |
| H (micron) | 0.80 | 0.80 | | | 0.35 | 0.35 |
| L/W/H accuracy (micron) | 0.10 | 0.10 | | | 0.10 | 0.10 |
| Size uniformity at 95% ± % | 4% | 4% | | | 13% | 11% |
| L/W | 1.3 | 1.3 | | | 1.1 | 1.3 |
| L/H | 2.9 | 2.9 | | | 2.3 | 2.6 |

TABLE 10

| | Example | | | | | |
|---|---|---|---|---|---|---|
| | 49 | 50 | 51 | 52 | 53 | 54 |
| Al/Si (molar) | 0.004 | 0.004 | 0.004 | 0.004 | 0.004 | 0.004 |
| TPA/Si (molar) | 0.125 | 0.125 | 0.125 | 0.125 | 0.125 | 0.125 |
| H₂O/Si (molar) | 50 | 50 | 50 | 50 | 50 | 50 |
| M/Si (molar) | 0.01 | 0.01 | 0.01 | 0.01 | 0.01 | 0.01 |
| OH⁻/Si (molar) | 0.135 | 0.135 | 0.135 | 0.135 | 0.135 | 0.135 |
| T of step (e) (° C.) | 25 | 25 | 25 | 35 | 50 | 50 |
| Time of step (e) (h) | 12 | 12 | 24 | 12 | 12 | 24 |
| T of step (g) (° C.) | 160 | 160 | 160 | 160 | 160 | 160 |
| Time of step (h) (h) | 40 | 40 | 40 | 40 | 40 | 40 |
| Heating rate (° C./h) | 100 | 200 | 100 | 100 | 100 | 100 |
| Stirring rate (RPM) | 350 | 350 | 350 | 350 | 350 | 350 |
| XRD | MFI | MFI | MFI | MFI | MFI | MFI |
| D50 (micron) | 1.6 | 2.1 | 0.6 | 1.6 | 1.3 | 0.9 |
| Span | 0.8 | 0.8 | 1.0 | 0.8 | 0.8 | 0.8 |
| QPM uniformity | 98% | 98% | 98% | 98% | 98% | 98% |
| Twinning | 2% | 2% | 2% | 2% | 2% | 2% |
| L (micron) | 2.60 | 3.00 | 0.85 | 2.50 | 2.10 | 1.40 |
| W (micron) | 1.50 | 1.90 | 0.60 | 1.70 | 1.30 | 1.00 |
| H (micron) | 0.70 | 0.80 | 0.30 | 0.70 | 0.55 | 0.40 |
| L/W/H accuracy (micron) | 0.10 | 0.10 | 0.10 | 0.10 | 0.10 | 0.10 |
| Size uniformity at 95% ± % | 4% | 3% | 12% | 4% | 5% | 7% |
| L/W | 1.7 | 1.6 | 1.4 | 1.5 | 1.6 | 1.4 |
| L/H | 3.7 | 3.8 | 2.8 | 3.6 | 3.8 | 3.5 |

### Examples 55-64

Examples 55-64 were prepared with the following procedure:
(a) aqueous TPAOH was added to TEOS under stirring;
(b) the mixture of step (a) was stirred for 1 hour at 25° C.;
(c) an aqueous solution composed of 10 wt % NaOH and 7.8 wt % Al(OH)₃ (aqueous aluminum sulfate was used for Examples 56-58) was added slowly under stirring;
(d) water was added to the mixture of step (c);
(e) the mixture of step (d) was stirred at 25° C. for different amount of time; then
(f) the mixture of step (e) was transferred to a stirred autoclave;
(g) the autoclave was heated to 160° C. with 100° C./h heating rate;
(h) the autoclave was maintained at 160° C. for 40 hours with 350 RPM stirring rate;
(i) the autoclave was cooled down to 25° C. and the crystals were recovered and washed using centrifugation or filtration, and dried at 60° C. for 24 hours; and
(j) the product of step (i) was analyzed by XRD, laser scatting, and SEM.

The following Tables 11-12 summarized the slurry composition, crystallization conditions, and product characterization results, such as quasi parallelepiped morphology (QPM) and hexagonal morphology (HEX).

TABLE 11

| | Example | | | | |
|---|---|---|---|---|---|
| | 55 | 56 | 57 | 58 | 59 |
| Al/Si (molar) | 0.002 | 0.008 | 0.002 | 0.004 | 0.008 |
| TPA/Si (molar) | 0.125 | 0.125 | 0.125 | 0.125 | 0.125 |
| H₂O/Si (molar) | 50 | 50 | 50 | 50 | 50 |
| M/Si (molar) | 0.005 | 0.02 | 0 | 0 | 0 |
| OH⁻/Si (molar) | 0.13 | 0.145 | 0.125 | 0.125 | 0.125 |
| T of step (e) (° C.) | 25 | 25 | 25 | 25 | 25 |
| Time of step (e) (h) | 12 | 12 | 12 | 12 | 12 |
| T of step (g) (° C.) | 160 | 160 | 160 | 160 | 160 |
| Time of step (h) (h) | 40 | 40 | 40 | 40 | 40 |
| Heating rate (° C./h) | 100 | 100 | 100 | 100 | 100 |
| Stirring rate (RPM) | 350 | 350 | 350 | 350 | 350 |
| XRD | MFI | MFI | MFI | MFI | MFI |
| D50 (micron) | 1.9 | 1.7 | 2.0 | 3.5 | 5.0 |
| Span | 1.0 | 0.9 | 1.0 | 1.5 | 1.6 |
| QPM uniformity | 98% | 98% | 98% | 98% | 95% |
| Twinning | 2% | 2% | 2% | 2% | 5% |
| L (micron) | 2.80 | 2.60 | 3.00 | 3.00 | |
| W (micron) | 1.70 | 1.70 | 1.80 | 2.00 | |
| H (micron) | 0.75 | 0.70 | 0.70 | 0.80 | |
| L/W/H accuracy | 0.10 | 0.10 | 0.10 | 0.20 | |
| (micron) | | | | | |
| Size uniformity at | 4% | 4% | 3% | 7% | |
| 95% ± % | | | | | |
| L/W | 1.6 | 1.5 | 1.7 | 1.5 | |
| L/H | 3.7 | 3.7 | 4.3 | 3.8 | |

TABLE 12

| | Example | | | | |
|---|---|---|---|---|---|
| | 60 | 61 | 62 | 63 | 64 |
| Al/Si (molar) | 0.004 | 0.004 | 0.004 | 0.004 | 0.004 |
| TPA/Si (molar) | 0.1 | 0.075 | 0.125 | 0.125 | 0.125 |
| H₂O/Si (molar) | 50 | 50 | 50 | 50 | 50 |
| M/Si (molar) | 0.01 | 0.01 | 0.01 | 0.01 | 0.01 |
| OH⁻/Si (molar) | 0.11 | 0.085 | 0.135 | 0.135 | 0.135 |
| T of step (e) (° C.) | 25 | 25 | 25 | 25 | 25 |
| Time of step (e) (h) | 12 | 12 | 8 | 4 | 0 |
| T of step (g) (° C.) | 160 | 160 | 160 | 160 | 160 |
| Time of step (h) (h) | 40 | 40 | 40 | 40 | 40 |
| Heating rate (° C./h) | 100 | 100 | 100 | 100 | 100 |
| Stirring rate (RPM) | 350 | 350 | 350 | 350 | 350 |
| XRD | MFI | MFI | MFI | MFI | MFI |
| D50 (micron) | 4.3 | 11.5 | 1.7 | 2.0 | 1.9 |
| Span | 1.1 | 1.6 | 0.9 | 0.8 | 0.9 |
| QPM uniformity | 95% | | 98% | 98% | 98% |
| Twinning | 5% | | 2% | 2% | 2% |
| L (micron) | | | 2.70 | 3.10 | 2.70 |
| W (micron) | | | 1.80 | 1.90 | 1.80 |
| H (micron) | | | 0.70 | 0.75 | 0.75 |
| L/W/H accuracy | | | 0.10 | 0.10 | 0.10 |
| (micron) | | | | | |
| Size uniformity at | | | 4% | 3% | 4% |
| 95% ± % | | | | | |
| L/W | | | 1.5 | 1.6 | 1.5 |
| L/H | | | 3.9 | 4.1 | 3.6 |

### Examples 65-81

Examples 65-81 were prepared with the following procedure:
(a) TEOS was added to aqueous TPAOH under stirring;
(b) the mixture of step (a) was stirred for 1 hour at 25° C.;
(c) an aqueous solution composed of 10 wt % NaOH and 7.8 wt % Al(OH)₃ was added slowly under stirring;
(d) water was added to the mixture of step (c);
(e) the mixture of step (d) was stirred at 25° C. for 12 hours; then
(f) the mixture of step (e) was transferred to a stirred autoclave;
(g) the autoclave was heated to 160° C. with 100° C./h heating rate;
(h) the autoclave was maintained at 160° C. for 40 hours with 350 RPM stirring rate;
(i) the autoclave was cooled down to 25° C. and the crystals were recovered and washed using centrifugation or filtration, and dried at 60° C. for 24 hours; and
(j) the product of step (i) was analyzed by XRD, laser scatting, and SEM.

The following Tables 13-15 summarized the slurry composition, crystallization conditions, and product characterization results, such as quasi parallelepiped morphology (QPM) and hexagonal morphology (HEX).

TABLE 13

| | Example | | | | | |
|---|---|---|---|---|---|---|
| | 65 | 66 | 67 | 68 | 69 | 70 |
| Al/Si (molar) | 0 | 0 | 0 | 0 | 0 | 0 |
| TPA/Si (molar) | 0.075 | 0.1 | 0.175 | 0.25 | 0.35 | 0.5 |
| H₂O/Si (molar) | 50 | 50 | 50 | 50 | 50 | 50 |
| M/Si (molar) | 0 | 0 | 0 | 0 | 0 | 0 |
| OH⁻/Si (molar) | 0.075 | 0.1 | 0.175 | 0.25 | 0.35 | 0.5 |
| Heating rate (° C./h) | 100 | 100 | 100 | 100 | 100 | 100 |
| Stirring rate (RPM) | 350 | 350 | 350 | 350 | 350 | 350 |
| XRD | MFI | MFI | MFI | MFI | MFI | MFI |
| D50 (micron) | 6.1 | 4.8 | 0.8 | 0.5 | 0.4 | 1.0 |
| Span | 1.7 | 1.3 | 2.1 | 3.9 | 56.1 | 1.7 |
| QPM uniformity | 25% | 80% | 95% | 95% | 95% | HEX |
| Twinning | 75% | 20% | 5% | 5% | 5% | 0% |
| L (micron) | | | 0.90 | 0.40 | 0.25 | |
| W (micron) | | | 0.75 | 0.35 | 0.25 | |
| H (micron) | | | 0.35 | 0.20 | 0.15 | |
| L/W/H accuracy | | | 0.05 | 0.05 | 0.05 | |
| (micron) | | | | | | |
| Size uniformity at | | | 6% | 13% | 20% | |
| 95% ± % | | | | | | |
| L/W | | | 1.2 | 1.1 | 1.0 | |
| L/H | | | 2.6 | 2.0 | 1.7 | |

TABLE 14

| | Example | | | |
|---|---|---|---|---|
| | 71 | 72 | 73 | 74 |
| Al/Si (molar) | 0.004 | 0.004 | 0.004 | 110.004 |
| TPA/Si (molar) | 0.175 | 0.25 | 0.35 | 0.5 |
| H₂O/Si (molar) | 50 | 50 | 50 | 50 |
| M/Si (molar) | 0.01 | 0.01 | 0.01 | 0.01 |
| OH⁻/Si (molar) | 0.185 | 0.26 | 0.36 | 0.51 |
| Heating rate (° C./h) | 100 | 100 | 100 | 100 |
| Stirring rate (RPM) | 350 | 350 | 350 | 350 |
| XRD | MFI | MFI | MFI | MFI |
| D50 (micron) | 1.1 | 0.7 | 0.3 | 0.9 |
| Span | 2.0 | 2.1 | 1.1 | 412.8 |
| QPM uniformity | 95% | 95% | 95% | 0% |
| Twinning | 5% | 5% | 5% | |
| L (micron) | 0.85 | 0.35 | 0.30 | |
| W (micron) | 0.70 | 0.30 | 0.25 | |
| H (micron) | 0.35 | 0.20 | 0.15 | |
| L/W/H accuracy (micron) | 0.05 | 0.05 | 0.05 | |
| Size uniformity at 95% ± % | 6% | 14% | 17% | |
| L/W | 1.2 | 1.2 | 1.2 | |
| L/H | 2.4 | 1.8 | 2.0 | |

TABLE 15

| | Example | | | | | | |
|---|---|---|---|---|---|---|---|
| | 75 | 76 | 77 | 78 | 79 | 80 | 81 |
| Al/Si (molar) | 0.01 | 0.01 | 0.01 | 0.01 | 0.01 | 0.01 | 0.01 |
| TPA/Si (molar) | 0.075 | 0.1 | 0.125 | 0.175 | 0.25 | 0.35 | 0.5 |
| H₂O/Si (molar) | 50 | 50 | 50 | 50 | 50 | 50 | 50 |
| M/Si (molar) | 0.025 | 0.025 | 0.025 | 0.025 | 0.025 | 0.025 | 0.025 |
| OH⁻/Si (molar) | 0.1 | 0.125 | 0.15 | 0.2 | 0.275 | 0.375 | 0.525 |
| Heating rate (° C./h) | 100 | 100 | 100 | 100 | 100 | 100 | 100 |
| Stirring rate (RPM) | 350 | 350 | 350 | 350 | 350 | 350 | 350 |
| XRD | MFI | MFI | MFI | MFI | MFI | MFI | MFI |
| D50 (micron) | 7.7 | 2.7 | 1.4 | 0.6 | 0.5 | 0.6 | 1.4 |
| Span | 1.1 | 1.1 | 1.0 | 0.9 | 1.8 | 2.9 | 1.6 |
| QPM uniformity | 75% | 90% | 95% | 95% | 95% | 95% | 0% |
| Twinning | 25% | 10% | 5% | 5% | 5% | 5% | |
| L (micron) | | | 1.90 | 0.70 | 0.40 | 0.25 | |
| W (micron) | | | 1.30 | 0.60 | 0.40 | 0.25 | |
| H (micron) | | | 0.60 | 0.30 | 0.20 | 0.15 | |
| L/W/H accuracy (micron) | | | 0.10 | 0.05 | 0.05 | 0.05 | |
| Size uniformity at 95% ± % | | | 5% | 7% | 13% | 20% | |
| L/W | | | 1.5 | 1.2 | 1.0 | 1.0 | |
| L/H | | | 3.2 | 2.3 | 2.0 | 1.7 | |

### Examples 82-92

Examples 82-92 were prepared with the following procedure:
(a) TEOS was added to aqueous TPAOH under stirring;
(b) the mixture of step (a) was stirred for 1 hour at 25° C.;
(c) an aqueous solution composed of 10 wt % NaOH and 7.8 wt % Al(OH)₃ was added slowly under stirring;
(d) water was added to the mixture of step (c);
(e) the mixture of step (d) was stirred at 25° C. for 12 hours; then
(f) the mixture of step (e) was transferred to a stirred autoclave;
(g) the autoclave was heated to 190° C. with different heating rates;
(h) the autoclave was maintained at 190° C. for 40 hours with different stirring rates;
(i) the autoclave was cooled down to 25° C. and the crystals were recovered and washed using centrifugation or filtration, and dried at 60° C. for 24 hours; and
(j) the product of step (i) was analyzed by XRD, laser scatting, and SEM.

The following Tables 16-17 summarized the slurry composition, crystallization conditions, and product characterization results, such as quasi parallelepiped morphology (QPM) and hexagonal morphology (HEX).

TABLE 16

| | Example | | | | | |
|---|---|---|---|---|---|---|
| | 82 | 83 | 84 | 85 | 86 | 87 |
| Al/Si (molar) | 0.004 | 0.004 | 0.004 | 0.004 | 0.004 | 0.004 |
| TPA/Si (molar) | 0.125 | 0.125 | 0.125 | 0.125 | 0.125 | 0.125 |
| H₂O/Si (molar) | 50 | 50 | 50 | 50 | 50 | 50 |
| M/Si (molar) | 0.01 | 0.01 | 0.01 | 0.01 | 0.01 | 0.01 |
| OH⁻/Si (molar) | 0.135 | 0.135 | 0.135 | 0.135 | 0.135 | 0.135 |
| Heating rate (° C./h) | 10 | 20 | 50 | 100 | 10 | 20 |
| Stirring rate (RPM) | 50 | 50 | 50 | 50 | 150 | 150 |
| XRD | MFI | MFI | MFI | MFI | MFI | MFI |
| D50 (micron) | 0.9 | 1.2 | 1.1 | 1.7 | 0.8 | 0.8 |
| Span | 1.9 | 1.8 | 1.3 | 1.2 | 1.5 | 1.3 |
| QPM uniformity | 50% | 90% | 90% | 95% | 75% | 90% |
| Twinning | 50% | 10% | 10% | 5% | 25% | 10% |
| L (micron) | | | | 2.00 | | |
| W (micron) | | | | 1.00 | | |
| H (micron) | | | | 0.50 | | |
| L/W/H accuracy (micron) | | | | 0.20 | | |
| Size uniformity at 95% ± % | | | | 10% | | |
| L/W | | | | 2.0 | | |
| L/H | | | | 4.0 | | |

TABLE 17

| | Example | | | | |
|---|---|---|---|---|---|
| | 88 | 89 | 90 | 91 | 92 |
| Al/Si (molar) | 0.004 | 0.004 | 0.004 | 0.004 | 0.004 |
| TPA/Si (molar) | 0.125 | 0.125 | 0.125 | 0.125 | 0.125 |
| H₂O/Si (molar) | 50 | 50 | 50 | 50 | 50 |
| M/Si (molar) | 0.01 | 0.01 | 0.01 | 0.01 | 0.01 |
| OH⁻/Si (molar) | 0.135 | 0.135 | 0.135 | 0.135 | 0.135 |
| Heating rate (° C./h) | 50 | 100 | 10 | 20 | 50 |
| Stirring rate (RPM) | 150 | 150 | 350 | 350 | 350 |
| XRD | MFI | MFI | MFI | MFI | MFI |
| D50 (micron) | 0.8 | 1.2 | 0.9 | 1.1 | 1.6 |
| Span | 1.3 | 1.4 | 1.3 | 1.2 | 0.9 |
| QPM uniformity | 95% | 95% | 90% | 90% | 95% |
| Twinning | 5% | 5% | 10% | 10% | 5% |
| L (micron) | 1.00 | 1.10 | | | 2.00 |
| W (micron) | 0.70 | 0.75 | | | 1.20 |
| H (micron) | 0.30 | 0.40 | | | 0.50 |
| L/W/H accuracy | 0.10 | 0.10 | | | 0.10 |
| (micron) | | | | | |
| Size uniformity at | 10% | 9% | | | 5% |
| 95% ± % | | | | | |
| L/W | 1.4 | 1.5 | | | 1.7 |
| L/H | 3.3 | 2.8 | | | 4.0 |

### Examples 93-100

Examples 93-100 were prepared with the following procedure:
(a) TEOS was added to aqueous TPAOH under stirring;
(b) the mixture of step (a) was stirred for 1 hour at 25° C.;
(c) water was added to the mixture of step (b);
(d) the mixture of step (c) was stirred at 25° C. for 12 hours; then
(e) the mixture of step (d) was transferred to a stirred autoclave;
(f) the autoclave was heated to 160° C. with 30° C./h heating rate;
(g) the autoclave was maintained at 160° C. for 40 hours with 350 RPM stirring rate;
(h) the autoclave was cooled down to 25° C. and the crystals were recovered and washed using centrifugation or filtration, and dried at 60° C. for 24 hours; and
(i) the product of step (h) was analyzed by XRD, laser scatting, and SEM.

The following Table 18 summarized the slurry composition, crystallization conditions, and product characterization results, such as quasi parallelepiped morphology (QPM) and hexagonal morphology (HEX).

TABLE 18

| | Example | | | | | | | |
|---|---|---|---|---|---|---|---|---|
| | 93 | 94 | 95 | 96 | 97 | 98 | 99 | 100 |
| Al/Si (molar) | 0 | 0 | 0 | 0 | 0 | 0 | 0 | 0 |
| TPA/Si (molar) | 0.2 | 0.2 | 0.2 | 0.2 | 0.2 | 0.2 | 0.2 | 0.2 |
| H₂O/Si (molar) | 10 | 15 | 20 | 30 | 45 | 60 | 80 | 100 |
| M/Si (molar) | 0 | 0 | 0 | 0 | 0 | 0 | 0 | 0 |
| OH⁻/Si (molar) | 0.2 | 0.2 | 0.2 | 0.2 | 0.2 | 0.2 | 0.2 | 0.2 |
| Heating rate | 30 | 30 | 30 | 30 | 30 | 30 | 30 | 30 |
| (° C./h) | | | | | | | | |
| Stirring rate | 350 | 350 | 350 | 350 | 350 | 350 | 350 | 350 |
| (RPM) | | | | | | | | |
| XRD | MFI | MFI | MFI | MFI | MFI | MFI | MFI | MFI |
| D50 (micron) | 0.1 | 0.1 | 0.1 | 0.2 | 0.4 | 0.4 | 0.6 | 0.7 |
| Span | 1.5 | 2.1 | 2.7 | 2.4 | 1.4 | 1.6 | 1.9 | 2.9 |
| QPM | | | | 95% | 95% | 95% | 95% | 95% |
| uniformity | | | | | | | | |

While the present invention has been described and illustrated by reference to particular embodiments, those of ordinary skill in the art will appreciate that the invention lends itself to many different variations not illustrated herein. For these reasons, then, reference should be made solely to the appended claims for purposes of determining the scope of the present invention.

What is claimed is:

1. A crystalline molecular sieve having MFI topology comprises a uniformity of crystal size of 90%±10% and at least 80% of the crystals having quasi parallelepiped morphology as measured by SEM.

2. The molecular sieve of claim 1, wherein said molecular sieve has less than 20% crystal twinning as measured by SEM.

3. The molecular sieve of claim 2, wherein said molecular sieve has a flatness of less than 0.05 as measured by SEM.

4. The molecular sieve of claim 1, wherein said molecular sieve has flatness of less than 0.02 as measured by SEM.

5. The molecular sieve of claim 4, wherein the length of the molecular sieve crystal is at least 1 micrometer.

6. The molecular sieve of claim 1, wherein the length/width ratio of the molecular sieve crystal is in the range of 1 to 10 as measured by SEM and the length/height ratio of the molecular sieve crystal is in the range of 2 to 20 as measured by SEM.

7. The molecular sieve of claim 1, wherein said crystalline molecular sieve is an aluminosilicate ZSM-5 molecular sieve.

8. The molecular sieve of claim 1, wherein said crystalline molecular sieve crystals have a span of less than 3 measured by laser scattering.

9. The molecular sieve of claim 1, wherein said crystalline molecular sieve has less than 5% crystal twinning.

10. A method of making a crystalline molecular sieve of any preceding claim comprising:
(a) providing a mixture comprising at least one source of at least one tetravalent element (Y), at least one source of hydroxide ion, at least one directing-agent (R), water, said mixture having the following molar composition:
H₂O/Y=10 to 1000
OH⁻/Y=0.001 to 2
R/Y=0.001 to 2
wherein R comprises at least one of TPAOH, TPACl, TPABr, TPAI, and TPAF, wherein OH⁻/Y is not corrected for trivalent ion;
(b) submitting the mixture at crystallization conditions to form a product comprising the crystalline molecular sieve, wherein the crystallization conditions comprise a temperature in the range of from 120° C. to 250° C., a crystallization time from about 1 hour to 200 hours; a heating rate in the range from at least 20° C./h, and a stirring speed at least 50 RPM; and
(c) recovering the molecular sieve.

11. The method of claim 10, wherein said mixture of step (a) further comprising at least one source of at least one metal element (M), and the molar ratio M/Y is in the range of from 0 to 0.5.

12. The method of claim 11, wherein said mixture of step (a) further comprising at least one source of at least one trivalent element (X), and the molar ratio Y/X₂ is in the range of from 10 to infinity.

13. The method of any one of claim 10, wherein the stirring speed is less than 600 RPM.

14. The method of any one of claim 13, wherein the stirring speed is in the range of 50-350 RPM.

15. A process for hydrocarbon conversion, comprising the step of:
contacting a hydrocarbon feedstock with the crystalline molecular sieve recited in claim 1, under conversion conditions to form a conversion product.
